SEQUENCE LISTING (1 sequence)

SEQ ID NO: 1 - DNA, 1642 bp, Streptococcus thermophilus
  source 1..1642: mol_type = genomic DNA
agaaagtatgatctgcttctacaccaaaaacttctttaaacttttctcttaactgtgatgtattcatagaatgtatctcacttattctttttgtttatactgaaattgtaaccactttcacatggaaaatcaatatttttagatactgtcaataattatgtgtaaacactcaagtagagttgaagaatgttaatcaaataagctttcaagtgtgtcagcacattggccaaaccctttatggatgcgttgattttgcttgaaattataatcttcaaacagggtaactaaataacgttccagagcctcctcgttaggaaaaagaaccttctttttcgtttgacgtttgatttctttgttaagagactcaatgaggtttgtcgaataaatgctatgccaaatctggtagggaaactgataaaaagttaaaagattatccgtattctccagactttccatgactttcctatactttggtttccattcggcgataaagttctctaaagcttgcactgccatttctaaattttcagcacgataaatcgttttaaattgctccagaataaccgctctatctgctcgtttcactttactagctagatttcgactaatatgaattaagcaacgttgttgtttagctaatgggtaagcctgattgataatctcttcaagccccttgaagccatcggtcactacaagagaaacctgttggattccttggttttgaagcttgtctaacagggtggaccaagaagcattgttttcatttggggcgatttcatatccaagaacagccttctgtccttctggtgtaatgccaagtgcgatatgaatacattctttactaacggttccacgtcttaatggaagataggttccgtcaagaaataaaacagagtaattggcttctaagcttcgctcatgaaaagtagcgacattctcctgagttgcttttgagatattagaaattgtggcaggactatagtgatgaccatacattcgctcgatgatatcactaatttctcgagtcgttacaccggtttgatagagtttgataaccatctcttccaagtggtcatctcgacgtccataagcgggaagcaaagctggactaaagttcccattacgatctctaggaatactcaactgaacagtcccatatttggtttcgaatttccgtgcatagcttccgttacgactattcccagaattatagcctaatttatcgtaaggttcataccctaaaaaggctgataactctgcttgaagcagatcattcatagctgtttcaagagaagtacggaaaaattcatcaatatcttgcttttgggctaggaagttaagtagttctgtggtaaactgagtcataggaataaatctctttctagtaatgttttgcaactctactataacggatttattcctttttgtgtttacacaacttattttacactacctattttatttttttagtaaaatataggtaaaaaataaaagttatgttatactgaaatatgaggaggatactatggctacattagcagatatcgcaaaattagcaggtgtatctatttcaactgtttcacgtgttcttaataaagatgaaactctttccgtaacagaggatactagacatcggatatta

(19) United States
(12) Patent Application Publication
(10) Pub. No.: US 2026/0223874 A1
(43) Pub. Date: Aug. 6, 2026

(54) Method for Producing Fermented Milk Products

(71) Applicant: CHR. HANSEN A/S, Hoersholm (DK)

(72) Inventors: Sabrina SALTAJI, Arpajon (FR); Nanna Skov HOEJLUND, Hoersholm (DK)

(73) Assignee: CHR. HANSEN A/S, Hoersholm (DK)

(21) Appl. No.: 19/147,601

(22) PCT Filed: Jan. 17, 2024
(86) PCT No.: PCT/EP2024/050963

§ 371 Date: Jul. 11, 2025

(30) Foreign Application Priority Data
Jan. 18, 2023 (EP) 23152202.0

(51) Int. Cl. A23C 9/123; C12N 1/20; C12R 1/225; C12R 1/46

(52) U.S. Cl. CPC A23C 9/1238; C12N 1/20; C12R 2001/225; C12R 2001/46

(57) ABSTRACT

The present disclosure is in the field of dairy technology. It relates to a method for producing a fermented milk product wherein the fermentation time is reduced, compositions, and fermented milk products obtained by the method or comprising the composition.

### FIELD

The present disclosure generally relates to methods of producing fermented milk products, in particular wherein the acidification time is reduced.

### BACKGROUND

Various methods of producing milk products such as fermented milk products are known and have been described. Time used for manufacturing of fermented milk products impacts the cost of such products. Therefore, there is a desire in the industry for reducing the time needed for manufacturing such as the fermentation time.

### SUMMARY

In a first aspect the present disclosure provides a method for producing a fermented milk product comprising the steps of:
(a) adding to a milk base a composition comprising (i) a first Streptococcus thermophilus strain deposited as DSM 34451 and/or (ii) a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element; and
(b) fermenting the milk base for a period of time until a target pH is reached;
wherein the fermentation time to reach the target pH when using the composition is reduced as compared to the fermentation time to reach the target pH of a benchmark starter culture without the DSM 34451 strain and/or comprising the galactose-negative mother strain.

In a second aspect the present disclosure provides a composition comprising the Streptococcus thermophilus strains DSM 34451 and a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element.

In a third aspect the present disclosure provides a fermented milk product obtainable by the method according to the disclosure above or comprising the composition according to the disclosure above.

### BRIEF DESCRIPTION OF THE SEQUENCES

SEQ ID NO: 1—galK DNA sequence from DSM 17876.

### DETAILED DESCRIPTION

### Method

The present disclosure relates to a method for producing a fermented milk product comprising the steps of:
(a) adding to a milk base a composition comprising (i) a first Streptococcus thermophilus strain deposited as DSM 34451 and/or (ii) a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element; and
(b) fermenting the milk base for a period of time until a target pH is reached;
wherein the fermentation time to reach the target pH when using the composition is reduced as compared to the fermentation time to reach the target pH of a benchmark starter culture without the DSM 34451 strain and/or comprising the galactose-negative mother strain.

As used herein the term “milk” is to be understood as the lacteal secretion obtained by milking any mammal, such as cows, sheep, goats, buffaloes or camels. In a preferred embodiment, the milk is cow's milk.

The term “milk base” may be any raw and/or processed milk material that can be subjected to fermentation according to the disclosed method. Thus, useful milk bases include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. The milk base may originate from any mammal, e.g. being substantially pure mammalian milk, or reconstituted milk powder or the milk base may originate from a plant material. Thus, a milk base may include protein/fat solutions made partly or exclusively of plant materials. Preferably, at least part of the protein in the milk base is (i) proteins naturally occurring in mammalian milk, such as casein or whey protein or (ii) proteins naturally occurring in plant milk. However, part of the protein may be proteins which are not naturally occurring in milk.

Prior to fermentation, the milk base may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk base to reduce or eliminate the presence of live organisms, such as microorganisms. Milk bases may be pasteurized prior to fermentation according to methods known in the art. Pasteurization may be attained by maintaining a specified temperature for a specified period of time. The specified temperature is usually attained by heating. Those of ordinary skill in the art are capable of selecting temperature and duration to kill or inactivate certain microorganisms. A rapid cooling step may follow.

The fermentation conditions are selected so as to support the achievement of the present invention, i.e. to obtain a fermented product such as a dairy or non-dairy product in solid or liquid form (fermented milk product).

In one embodiment the disclosure relates to a method, wherein the transposase insertion element is located in the galactose operon promotor region sequence corresponding to between nucleotides 133 and 1460 of SEQ ID No: 1.

In one embodiment the disclosure relates to a method, wherein the galactose operon promotor region sequence corresponds to nucleotides 105-133 and 1460-1491 of SEQ ID No: 1.

In one embodiment the disclosure relates to a method, wherein the transposase insertion element sequence corresponds to nucleotides 134-1459 of SEQ ID No: 1

In one embodiment the disclosure relates to a method, wherein the mother strain is DSM 17876

In one embodiment the disclosure relates to a method, wherein the second Streptococcus thermophilus strain is DSM 33677 and mutants and variants thereof.

In the present context, the term “galactose-positive S. thermophilus strain” or “gal-positive S. thermophilus strain” or “gal+S. thermophilus strain” as defined herein means that the pH was reduced by a value of at least 1.0 after 16 hours incubation at 37° C. in M17 with 2% galactose (galactose added as sole carbohydrate), inoculated in an amount of at least 10⁴ cells pr ml of M17. Selection of gal-positive strains is disclosed in WO2022/106418.

In the present invention “ability not to excrete galactose” means that the galactose positive strain is not able to excrete any measurable amount of galactose, in particular means that no measurable amount of galactose is detected when tested by assay II of WO2022/106418. In the present invention, “ability to excrete galactose but to consume the excreted galactose to completion at most 9 hours” means that the galactose positive strain is able to excrete galactose but to consume the excreted galactose to completion (i.e., to a level below the measurable amount) at most 9 hours after inoculation, in particular means that the galactose positive strain excretes galactose but consumes the excreted galactose to completion (i.e., to a level below the measurable amount) at most 9 hours after inoculation, when tested by assay II of WO2022/106418.

In an embodiment, the gal-positive S. thermophilus strain as defined herein is characterized by its ability not to excrete galactose, when inoculated at 1% (v/v) into a M17 medium supplemented with 0.5% (w/v) of lactose and incubated at 42° C., in particular when tested by assay II of WO2022/106418. In an embodiment, the gal-positive S. thermophilus strain as defined herein is characterized by its ability to excrete galactose but to consume the excreted galactose to completion at most 9 hours after being inoculated at 1% (v/v) into a M17 medium supplemented with 0.5% (w/v) of lactose and incubated at 42° C., in particular when tested by assay II of WO2022/106418. In an particular embodiment, the galactose-positive S. thermophilus strain as defined herein is characterized by its ability to excrete galactose but to consume the excreted galactose to completion at most 8 hours after being inoculated at 1% (v/v) into a M17 medium supplemented with 0.5% (w/v) of lactose and incubated at 42° C., in particular when tested by assay II of WO2022/106418.

In the present context, the term “mutant” should be understood as a strain derived, or a strain which can be derived from a strain of the invention (or the mother strain) by means of e.g. genetic engineering, radiation and/or chemical treatment. The mutant can also be a spontaneously occurring mutant. It is preferred that the mutant is a functionally equivalent mutant, e.g. a mutant that has substantially the same, or improved, properties (e.g. regarding viscosity, gel stiffness, mouth coating, flavor, post acidification, acidification speed, and/or phage robustness) as the mother strain. Such a mutant is a part of the present invention.

Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood one mutagenization step followed by a screening/selection step), but it is presently preferred that no more than 20, 10, or 5, treatments (or screening/selection steps) are carried out. In a presently preferred mutant less than 1%, less than 0.1%, less than 0.01%, less than 0.001% or less than 0.0001% of the nucleotides in the bacterial genome have been replaced with another nucleotide, or deleted, compared to the mother strain.

In the present context, the term “variant” should be understood as a strain which is functionally equivalent to a strain of the invention, e.g. having substantially the same, or improved, properties e.g. regarding viscosity, gel stiffness, mouth coating, flavor, post acidification, acidification speed, and/or phage robustness). Such variants, which may be identified using appropriate screening techniques, are a part of the present invention.

In one embodiment the disclosure relates to a method, further comprising Streptococcus thermophilus strains DSM 22935 and/or DSM 24655.

In one embodiment the disclosure relates to a method, wherein the starter culture further comprises one or more strains belonging to the species Lactobacillus delbrueckii subsp bulgaricus.

In one embodiment the disclosure relates to a method, wherein the one or more strains belonging to the species Lactobacillus delbrueckii subsp bulgaricus are selected from DSM 22586, DSM 24074, DSM 33571, and mutant and variants thereof.

In one embodiment the disclosure relates to a method, wherein reduction in the fermentation time is caused by a shorter lag phase and/or a shorter acidification time.

The lag phase is the initial phase of the fermentation. In the present disclosure the lag phase (Ta) was determined as the time in minutes from initial pH (start of fermentation) until at pH drop of 0.08 units were achieved. The acidification time was measured as the time in hours and minutes from end of lag phase until target pH was reached. The fermentation time is total time of the lag phase+acidification time.

The strains and combination of strains comprised in the method disclosed in this section may also be comprised in the compositions and/or fermented milk products disclosed in the following sections and vice versa.

### Composition

The present disclosure relates to a composition comprising the Streptococcus thermophilus strains DSM 34451 and a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element.

To ferment the milk base a composition such as a starter culture is added. In the present context the term “starter” or “starter culture” refers to a culture of one or more food-grade microorganisms in particular lactic acid bacteria, which are responsible for the acidification of the milk base and to assist the fermentation process in preparation of fermented products such as various foods, feeds and beverages. In the present context, a “yoghurt starter culture” is a bacterial culture which comprises at least one Lactobacillus delbrueckii subsp bulgaricus (L. bulgaricus) strain and at least one Streptococcus thermophilus (S. thermophilus) strain. In accordance herewith, a “yoghurt” refers to a fermented milk product obtainable by inoculating and fermenting a milk substrate with a composition comprising a L. bulgaricus strain and a S. thermophilus strain.

In one embodiment the disclosure relates to a composition, wherein the transposase insertion element is located in the galactose operon promotor region sequence corresponding to between nucleotides 133 and 1460 of SEQ ID No: 1.

In one embodiment the disclosure relates to a composition, wherein the galactose operon promotor region sequence corresponds to nucleotides 105-133 and 1460-1491 of SEQ ID No: 1.

In one embodiment the disclosure relates to a composition, wherein the transposase insertion element sequence corresponds to nucleotides 134-1459 of SEQ ID No: 1.

In one embodiment the disclosure relates to a composition, wherein the mother strain is DSM 17876.

In one embodiment the disclosure relates to a composition, wherein the second Streptococcus thermophilus strain is DSM 33677 and mutants and variants thereof.

In one embodiment the disclosure relates to a composition, further comprising one or more strains belonging to the species Lactobacillus delbrueckii subsp bulgaricus.

The composition according to claim 13, wherein the one or more Lactobacillus delbrueckii subsp bulgaricus strains are selected from DSM 22586, DSM 24074, DSM 33571, and mutant and variants thereof.

In one embodiment the disclosure relates to a composition comprising or consisting of the S. thermophilus strains DSM 34451 and DSM 33677 in combination with S. thermophilus strain DSM 22935 and Lactobacillus delbrueckii subsp bulgaricus strain DSM 22586. In one embodiment the disclosure relates to a composition comprising or consisting of the S. thermophilus strains DSM 34451 and DSM 33677 in combination with S. thermophilus strain DSM 22935 and Lactobacillus delbrueckii subsp bulgaricus strain DSM 24074. In one embodiment the disclosure relates to a composition comprising or consisting of the S. thermophilus strains DSM 34451 and DSM 33677 in combination with S. thermophilus strain DSM 22935 and Lactobacillus delbrueckii subsp bulgaricus strain DSM 33571.

In one embodiment the disclosure relates to a composition comprising or consisting of S. thermophilus strains DSM34451 and DSM 33677 in combination with S. thermophilus strain DSM 24655 and Lactobacillus delbrueckii subsp bulgaricus strain DSM 22586. In one embodiment the disclosure relates to a composition comprising or consisting of S. thermophilus strains DSM 34451 and DSM 33677 in combination with S. thermophilus strain DSM 24655 and Lactobacillus delbrueckii subsp bulgaricus strain DSM 24074. In one embodiment the disclosure relates to a composition comprising or consisting of S. thermophilus strains DSM34451 and DSM 33677 in combination with S. thermophilus strain DSM 24655 and Lactobacillus delbrueckii subsp bulgaricus strain DSM 33571.

In one embodiment the disclosure relates to a composition comprising from 10⁴ to 10¹² CFU (colony forming units)/g of the S. thermophilus strain, such as from 10⁵ to 10¹¹ CFU/g, such as from 10⁶ to 10¹⁰ CFU/g, or such as from 10⁷ to 10⁹ CFU/g of the S. thermophilus strain.

In one embodiment the disclosure relates to a composition further comprising from 10⁴ to 10¹² CFU/g of the L. bulgaricus strain, such as from 10⁵ to 10¹¹ CFU/g, such as from 10⁶ to 10¹⁰ CFU/g, or such as from 10⁷ to 10⁹ CFU/g of the L. bulgaricus strain.

L. bulgaricus, S. thermophilus and other lactic acid bacteria are commonly used as starter cultures serving a technological purpose in the production of various foods, such as in the dairy industry, such as for fermented milk products. Thus, in another preferred embodiment the composition is suitable as a starter culture. The composition may be a starter culture such as a yoghurt starter culture.

The composition may be provided in several forms. It may be a frozen form, dried form, spray-dried form, vacuum-dried form, air-dried, tray-dried, freeze dried form, or liquid form. It may appear as powder, pellets, tablets, or any combination thereof. Thus, in one embodiment the composition is in frozen, dried, freeze-dried or liquid form. Typically, the storage stability of food products can be extended by formulating the product with low water activity. By controlling the water activity (Aw), it is possible to predict and regulate the effect of moisture migration on the product. Therefore, it may be preferred that the water activity (Aw) of the dried compositions herein is in the range from 0.01-0.8, preferably in the range from 0.05-0.4.

The composition as disclosed herein may be in a form of a mixture or in a form of a kit-of-parts. The expression “mixture” means that the S. thermophilus strain(s) and the Lactobacillus delbrueckii subsp bulgaricus strain(s) are physically mixed together. In an embodiment, the S. thermophilus strain(s) and the Lactobacillus delbrueckii subsp bulgaricus strain(s) are in the same box or in the same pouch. In contrast, the expression “A kit-of-part” comprising S. thermophilus strain(s) and the Lactobacillus delbrueckii subsp bulgaricus strain means that the culture of the S. thermophilus strain(s) and the Lactobacillus delbrueckii subsp bulgaricus strain(s) culture are physically separated but intended to be used together. Thus, the culture of the S. thermophilus strain(s) and the Lactobacillus delbrueckii subsp bulgaricus strain(s) culture are in different boxes or sachets. In an embodiment, the culture of the S. thermophilus strain(s) and the Lactobacillus delbrueckii subsp bulgaricus strain(s) are under the same format, i.e., are in a frozen format, in the form of pellets or frozen pellets, a powder form, such as a dried or freeze-dried powder.

The composition may additionally comprise cryoprotectants, lyoprotectants, antioxidants, nutrients, fillers, flavorants or mixtures thereof. The composition may comprise one or more of cryoprotectants, lyoprotectants, antioxidants and/or nutrients. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the disclosure the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity e.g. cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i. e. the production of e. g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the composition of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the composition contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the milk base. It should be understood that ammonia may be added instead of ammonium salt. Thus, the term ammonium salt comprises ammonia (NH3), NH40H, NH4+, and the like.

In one embodiment the composition may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

### Fermented Milk Product

The present disclosure relates to a fermented milk product obtainable by the method according to the disclosure above or comprising the composition according to the disclosure above. Accordingly, the present disclosure relates to fermented milk products derived from any of the milk bases described above and comprising the specific lactic acid bacteria as disclosed herein.

In one embodiment the fermented milk product is a dairy product. The dairy product may be selected from the group consisting of but not limited to yoghurt, buttermilk, kefir or cheese such as but not limited to fresh cheese or pasta filata. In one embodiment the fermented product may be in the form of a stirred type product, a set type product or a drinkable product.

Although the fermented product and/or the dairy product itself comprise acid and flavor generated during fermentation it may be desired that fermented product and/or the dairy product comprises an ingredient selected from the group consisting of a fruit concentrate, a syrup, a probiotic bacterial culture, a coloring agent, a thickening agent, a flavoring agent, a preserving agent and combinations thereof.

Likewise, an enzyme may be added to the substrate e.g. the milk substrate before, during and/or after the fermenting, the enzyme being selected from the group consisting of an enzyme able to crosslink proteins, transglutaminase, an aspartic protease, chymosin, rennet and combinations thereof.

In one embodiment the disclosure relates to the use of (i) a first Streptococcus thermophilus strain deposited as DSM 34451 and/or (ii) a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element for producing a fermented milk product, wherein the fermentation time to reach the target pH when using the starter culture is reduced as compared to the fermentation time to reach the target pH of a benchmark starter culture without the DSM 34451 strain and/or comprising the galactose-negative mother strain.

### Probiotic Strains

The term “probiotic bacteria” refers to viable bacteria which are administered in adequate amounts to a consumer for the purpose of achieving a health-promoting effect in the consumer. Probiotic bacteria are capable of surviving the conditions of the gastrointestinal tract after ingestion and colonize the intestine of the consumer.

It will be appreciated that the Lactobacillus genus taxonomy was updated in 2020. The new taxonomy is disclosed in Zheng et al. 2020 (Zheng et al., ct. J. Syst. Evol. Microbiol. DOI 10.1099/ijsem.0.004107) and will be cohered to herein if nothing else is noticed. For the purpose of the present disclosure new and old names of some relevant Lactobacillus species are listed.

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei subsp. paracasei | Lacticaseibacillus paracasei subsp. Paracasei |
| Lactobacillus plantarum subsp. plantarum | Lactiplantibacillus plantarum subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

The probiotic bacteria may be used in the method disclosed herein. In one embodiment the disclosure relates to a method further comprising a step of adding one or more probiotic bacteria. Such probiotic bacteria may be added before, during or after fermentation of the milk base. In one embodiment the disclosure relates to a method wherein the probiotic bacteria are added before, during or after fermentation of the milk base. The probiotic bacteria may be comprised in a composition, e.g. a starter culture, or added separately. It is desirable for achieving a health-promoting effect that the fermented milk product comprises probiotic bacteria.

In one embodiment the disclosure relates to probiotic strains selected from the group consisting of bacteria of the genus Lactobacillus, such as Lactobacillus acidophilus, Lacticaseibacillus paracasei, Lacticaseibacillus rhamnosus, Lacticaseibacillus casei, Lactobacillus delbrueckii, Lactobacillus lactis, Lactiplantibacillus plantarum, Limosilactobacillus reuteri and Lactobacillus johnsonii, the genus Bifidobacterium, such as the Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis, and the like.

In one embodiment the disclosure relates to a probiotic Lactobacillus strain selected from the group consisting of Lactobacillus acidophilus, Lacticaseibacillus paracasei, Lacticaseibacillus rhamnosus, Lactobacillus casei, Lactobacillus delbrueckii, Lactobacillus lactis, Lactiplantibacillus plantarum, Limosilactobacillus reuteri and Lactobacillus johnsonii. In one embodiment the disclosure relates to a probiotic Lactobacillus strain selected from the group consisting of a Lacticaseibacillus rhamnosus strain and a Lacticaseibacillus paracasei strain. In one embodiment the disclosure relates to a probiotic strain is Lacticaseibacillus rhamnosus strain LGG® deposited as ATCC 53103. In one embodiment the disclosure relates to a probiotic strain is Lacticaseibacillus paracasei strain CRL 431 deposited as ATCC 55544.

In one embodiment the disclosure relates to a probiotic Bifidobacterium strain selected from the group consisting of Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis. In one embodiment the disclosure relates to a probiotic Bifidobacterium animalis subsp. lactis BB-12 strain deposited as DSM 15954.

The compositions of the present disclosure may further comprise other lactic acid bacteria such as but not limited to probiotic bacteria. Selection of the specific probiotic bacteria such as e.g. Bifidobacterium, Lactobacillus acidophilus and/or Lacticaseibacillus rhamnosus will depend on the fermented milk product to be produced. In one embodiment the disclosure relates to a composition further comprising lactic acid bacteria selected from the group consisting of Bifidobacterium such as Bifidobacterium animalis subsp. Lactis (e.g. BB-12®), Lactobacillus acidophilus (LA-5®), Lacticaseibacillus rhamnosus (e.g. LGG®) and combinations thereof.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1: The applicant has made the following deposits at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany.

| Strain | Accession No. | Deposit date |
|---|---|---|
| Streptococcus thermophilus | DSM 34451 | 2022 Dec. 8 |

### SEQUENCE LISTING

SEQ ID NO 1: DNA sequence from DSM 17876 covering
galK start, gal operon promoter with insertion
sequence and start of galR:
AGAAAGTATGATCTGCTTCTACACCAAAAACTTCTTTAAACTTTTCTCT
TAACTGTGATGTATTCATAGAATGTATCTCACTTATTCTTTTTGTTTAT
ACTGAAATTGTAACCACTTTCACATGGAAAATCAATATTTTTAGATACT
GTCAATAATTATGTGTAAACACTCAAGTAGAGTTGAAGAATGTTAATCA
AATAAGCTTTCAAGTGTGTCAGCACATTGGCCAAACCCTTTATGGATGC
GTTGATTTTGCTTGAAATTATAATCTTCAAACAGGGTAACTAAATAACG
TTCCAGAGCCTCCTCGTTAGGAAAAAGAACCTTCTTTTTCGTTTGACGT
TTGATTTCTTTGTTAAGAGACTCAATGAGGTTTGTCGAATAAATGCTAT
GCCAAATCTGGTAGGGAAACTGATAAAAAGTTAAAAGATTATCCGTATT
CTCCAGACTTTCCATGACTTTCCTATACTTTGGTTTCCATTCGGCGATA
AAGTTCTCTAAAGCTTGCACTGCCATTTCTAAATTTTCAGCACGATAAA
TCGTTTTAAATTGCTCCAGAATAACCGCTCTATCTGCTCGTTTCACTTT
ACTAGCTAGATTTCGACTAATATGAATTAAGCAACGTTGTTGTTTAGCT
AATGGGTAAGCCTGATTGATAATCTCTTCAAGCCCCTTGAAGCCATCGG
TCACTACAAGAGAAACCTGTTGGATTCCTTGGTTTTGAAGCTTGTCTAA
CAGGGTGGACCAAGAAGCATTGTTTTCATTTGGGGCGATTTCATATCCA
AGAACAGCCTTCTGTCCTTCTGGTGTAATGCCAAGTGCGATATGAATAC
ATTCTTTACTAACGGTTCCACGTCTTAATGGAAGATAGGTTCCGTCAAG
AAATAAAACAGAGTAATTGGCTTCTAAGCTTCGCTCATGAAAAGTAGCG
ACATTCTCCTGAGTTGCTTTTGAGATATTAGAAATTGTGGCAGGACTAT
AGTGATGACCATACATTCGCTCGATGATATCACTAATTTCTCGAGTCGT
TACACCGGTTTGATAGAGTTTGATAACCATCTCTTCCAAGTGGTCATCT
CGACGTCCATAAGCGGGAAGCAAAGCTGGACTAAAGTTCCCATTACGAT
CTCTAGGAATACTCAACTGAACAGTCCCATATTTGGTTTCGAATTTCCG
TGCATAGCTTCCGTTACGACTATTCCCAGAATTATAGCCTAATTTATCG
TAAGGTTCATACCCTAAAAAGGCTGATAACTCTGCTTGAAGCAGATCAT
TCATAGCTGTTTCAAGAGAAGTACGGAAAAATTCATCAATATCTTGCTT
TTGGGCTAGGAAGTTAAGTAGTTCTGTGGTAAACTGAGTCATAGGAATA
AATCTCTTTCTAGTAATGTTTTGCAACTCTACTATAACGGATTTATTCC
TTTTTGTGTTTACACAACTTATTTTACACTACCTATTTTATTTTTTTAG
TAAAATATAGGTAAAAAATAAAAGTTATGTTATACTGAAATATGAGGAG
GATACTATGGCTACATTAGCAGATATCGCAAAATTAGCAGGTGTATCTA
TTTCAACTGTTTCACGTGTTCTTAATAAAGATGAAACTCTTTCCGTAAC
AGAGGATACTAGACATCGGATATTA.

Start nucleotide galK gene (minus strand): 67
Transposase encoding Insertion sequence includes nucleotides: 134-1459
Start nucleotide galR gene (plus strand): 1526
Promoter region on both sides of insertion element (IS element sits in the middle):
Part 1: 105-133
Part 2: 1460-1491

### EXAMPLES

### Material & Methods

TABLE 2
Milk Bases

| | Milk Base A | Milk Base C |
|---|---|---|
| INGREDIENT: | Sweet | Plain |
| PROTEIN | 3.10% | 3.80% |
| FAT | 3.25% | 1.20% |
| SUGAR | 10.00% | — |

TABLE 3
Content of Lactic Acid Bacteria strains in Samples (S) and Benchmarks (B). The content
of the Streptococcus thermophilus (ST) strains DSM 17876 and DSM 34451 in S1-S8 and B2-B3
are indicated in the table relative to the content in B1 (1). The content of DSM 33677 in S1-S4,
S6-S8 and B1-B3 are indicated relative to the content in S5 (1).

| STRAIN | S1 | S2 | S3 | S4 | S5 | S6 | S7 | S8 | B1 | B2 | B3 |
|---|---|---|---|---|---|---|---|---|---|---|---|
| DSM 17876 | 1.3 | 1 | 0 | 0 | 0 | 0 | 0 | 2.5 | 1 | 0 | 1 |
| DSM 33677 | 0 | 0 | 3 | 2.7 | 1 | 1.8 | 1.3 | 0 | 0 | 0 | 0 |
| DSM 34451 | 0.8 | 2 | 2 | 2.4 | 3.6 | 0.8 | 2 | 2 | 1 | 0 | 1 |

The Lactobacillus delbrueckii subsp. bulgaricus (LB) strain DSM 22586 was present in all starter cultures in about equal amounts.

TABLE 4
Fermentation method

| STEP | METHOD 1 | METHOD 2 | METHOD 3 |
|---|---|---|---|
| Pre-heating | 70° C. | 70° C. | 70° C. |
| Heat treatment | 95° C. for 5 min | 95° C. for 5 min | 95° C. for 5 min |
| Homogenization | 200/40 bars at 70° C. | 200/40 bars at 70° C. | 200/40 bars at 70° C. |
| Inoculation rate | 20 U/100 L | 20 U/100 L | 20 U/100 L |
| Inoculation | Inoculation in 100 mL | Inoculation in 100 mL | Inoculation in 200 mL |
| temperature | glass cups at 6° C. | plastic cups at 45° C. | baby bottles at 40° C. |
| Waiting time before | Waiting time at 6° C. | NA | NA |
| heating | before heating to | | |
| | 45° C. | | |
| Fermentation | 40° C. | 40° C. | 40° C. |
| temperature | | | |
| Target pH | 4.65; 4.60; 4.55 | 4.65; 4.60; 4.55 | 4.70; 4.65 |
| pH measured at | 5 h; 6 h; 24 h | 5 h; 6 h; 24 h | NA |
| times | | | |

Acidification of a milk base inoculated with a starter culture Sample (S) or starter culture Benchmark (B) was determined by following the change of pH over time during fermentation at 40° C. pH measurements were continued until either Target pH or a defined time were reached. The samples were then cooled and stored at 6° C.

The Lag phase (Ta) was determined as time in minutes from initial pH (start of fermentation at 40° C.) until a pH drop of 0.08 unit was achieved.

### Example 1—Acidification Profile

Samples (S) and Benchmarks (B) were used to ferment Milk base A using Method 2 The tables below show that both the lag phase (Ta) as well as the fermentation time using the new samples (S2, S3, S4, S5) was reduced as compared to fermentation time using benchmark (B1, B2). All acidification curves stabilize about pH 4 after 24 hours of fermentation.

TABLE 5
Fermentation time to Target pH.

| Sample | Ta | TpH4.65 | TpH4.60 | TpH4.55 |
|---|---|---|---|---|
| S1 | 1 h 20 ± 2.4 | 5 h 05 ± 0.8 | 5 h 22 ± 0.9 | 5 h 39 ± 0.6 |
| S2 | 58.8 ± 0.9 | 4 h 27 ± 4.1 | 4 h 42 ± 4.8 | 4 h 58 ± 5.4 |
| S3 | 52.3 ± 0.1 | 4 h 34 ± 5.6 | 4 h 49 ± 6.0 | 5 h 07 ± 6.4 |
| S4 | 53.3 ± 3.6 | 4 h 20 ± 10.5 | 4 h 34 ± 11.5 | 4 h 49 ± 12.6 |
| S5 | 50.9 ± 1.4 | 4 h 08 ± 11.5 | 4 h 22 ± 12.9 | 4 h 38 ± 15.1 |
| B1 | 1 h 03 ± 3.1 | 4 h 46 ± 2.3 | 5 h 02 ± 2.7 | 5 h 19 ± 2.9 |
| B2 | 1 h 20 ± 0.1 | 4 h 48 ± 0.5 | 5 h 07 ± 0.1 | 5 h 29 ± 0.8 |

TABLE 6
pH at different time points after start of fermentation.

| Sample | pH after 5 h | pH after 6 h | pH after 24 h |
|---|---|---|---|
| S1 | 4.67 ± 0 | 4.50 ± 0 | 4.02 ± 0.03 |
| S2 | 4.54 ± 0.01 | 4.40 ± 0.01 | 3.98 ± 0.01 |
| S3 | 4.57 ± 0.01 | 4.44 ± 0.01 | 4.04 ± 0.01 |
| S4 | 4.52 ± 0.04 | 4.39 ± 0.03 | 4.00 ± 0.03 |
| S5 | 4.49 ± 0.05 | 4.36 ± 0.06 | 3.98 ± 0.08 |
| B1 | 4.61 ± 0.01 | 4.45 ± 0.01 | 3.99 ± 0.01 |
| B2 | 4.62 ± 0.01 | 4.49 ± 0 | 4.01 ± 0.01 |

### Example 2—Effect of Waiting Time on Fermentation Time

Samples and Benchmark were used to ferment Milk Base C using Method 1. Waiting time for the inoculated samples were 1, 24, or 30 hours before heating to 45° C. and transfer to the fermentation temperature of 40° C.

The tables below show that both the lag phase (Ta) as well as the fermentation time using the new samples (S3, S4) was reduced as compared to fermentation time using benchmark (B1).

TABLE 7
Fermentation time to Target pH.

| | Waiting | | | | |
|---|---|---|---|---|---|
| SAMPLE | time | Ta | TpH4.65 | TpH4.60 | TpH4.55 |
| B1 | 1 h | 65.22 | 337.5 | 362.0 | 391.5 |
| S3 | 1 h | 40.16 | 287.6 | 310.9 | 339.1 |
| S4 | 1 h | 43.10 | 280.6 | 303.8 | 332.7 |
| B1 | 24 h | 64.79 | 356.3 | 378.9 | 404.9 |
| S3 | 24 h | 40.06 | 276.9 | 296.0 | 318.1 |
| S4 | 24 h | 50.28 | 275.5 | 293.0 | 314.4 |
| B1 | 30 h | 82.53 | 330.1 | 349.9 | 373.0 |
| S3 | 30 h | 69.03 | 275.0 | 292.6 | 313.1 |
| S4 | 30 h | 76.72 | 276.0 | 292.7 | 311.7 |

TABLE 8
pH at different time points after start of fermentation.

| | Waiting | pH after | pH after | pH after |
|---|---|---|---|---|
| SAMPLE | time | 5 h | 6 h | 24 h |
| B1 | 1 h | 4.74 | 4.60 | 4.20 |
| S3 | 1 h | 4.62 | 4.52 | 4.19 |
| S4 | 1 h | 4.61 | 4.51 | 4.24 |
| B1 | 24 h | 4.80 | 4.64 | 4.09 |
| S3 | 24 h | 4.59 | 4.49 | 4.13 |
| S4 | 24 h | 4.58 | 4.49 | 4.13 |
| B1 | 30 h | 4.74 | 4.58 | 4.10 |
| S3 | 30 h | 4.58 | 4.46 | 4.13 |
| S4 | 30 h | 4.58 | 4.45 | 4.14 |

### Example 3

Samples (S) and Benchmarks (B) were used to ferment Milk Base A using Method 3.

The table below shows that fermentation time using the new samples was reduced as compared to fermentation time using benchmarks.

TABLE 9
Fermentation time to Target pH.

| SAMPLE | Ta | TpH4.70 | TpH4.65 |
|---|---|---|---|
| S1 | 78 | 5.06 | 5.27 |
| S2 | 80 | 5.20 | 5.37 |
| S3 | 74 | 4.49 | 5.07 |
| S6 | 70 | 5.16 | 5.13 |
| S7 | 78 | 5.03 | 5.32 |
| S8 | 72 | 5.13 | 5.33 |
| B1 | 80 | 5.40 | 5.33 |
| B3 | 88 | 5.37 | 5.51 |

1. A method for producing a fermented milk product comprising the steps of:
a) adding to a milk base a composition comprising (i) a first Streptococcus thermophilus strain deposited as DSM 34451 and/or (ii) a second Streptococcus thermophllus strain derived from a galactose-negative mother strain having a Transposase Insertion Element in the Galactose Operon Promoter Region, wherein said mutant strain becomes galactose-positive by the absence of the Transposase Insertion Element; and
b) fermenting the milk base for a period of time until a target pH is reached;
wherein the fermentation time to reach the target pH when using the composition is reduced as compared to the fermentation time to reach the target pH of a benchmark composition without the DSM 34451 strain and/or comprising the galactose-negative mother strain.

2. The method according to claim 1, wherein the transposase insertion element is located in the galactose operon promotor region sequence corresponding to between nucleotides 133 and 1460 of SEQ ID NO: 1.

3. The method according to claim 1, wherein the galactose operon promotor region sequence corresponds to nucleotides 105-133 and 1460-1491 of SEQ ID NO: 1.

4. The method according to claim 1, wherein the transposase insertion element sequence corresponds to nucleotides 134-1459 of SEQ ID NO: 1

5. The method according to claim 1, wherein the mother strain is DSM 17876.

6. The method according to claim 1, wherein the second Streptococcus thermophilus strain is DSM 33677 and mutants and variants thereof.

7. The method according to claim 1 further comprising Streptococcus thermophilus strains DSM 22935 and/or DSM 24655.

8. The method according to claim 1, wherein the composition further comprises one or more strains belonging to the species Lactobacillus delbrueckii subsp bulgaricus.

9. The method according to claim 8, wherein the one or more strains are selected from DSM 22586, DSM 24074, DSM 33571, and mutant and variants thereof.

10. The method according to claim 1, wherein reduction in the fermentation time is caused by a shorter lag phase and/or a shorter acidification time.

11. A composition comprising the Streptococcus thermophilus strains DSM 34451 and a second Streptococcus thermophilus strain derived from a galactose-negative mother strain having a Transposase Insertion Element in the Galactose Operon Promoter Region, wherein said mutant strain becomes galactose-positive by the absence of the Transposase Insertion Element.

12. The composition according to claim 11, wherein the second Streptococcus thermophilus strain is DSM 33677 and mutants and variants thereof.

13. The composition according to claim 11, further comprising one or more strains belonging to the species Lactobacillus delbrueckii subsp bulgaricus.

14. The composition according to claim 13, wherein the one or more Lactobacillus delbrueckii subsp bulgaricus strains are selected from DSM 22586, DSM 24074, DSM 33571, and mutant and variants thereof.

15. A fermented milk product produced obtainable by the method of claim 1.

16. A fermented milk product comprising the composition of claim 11.
